(19) United States
(12) Patent Application Publication
(10) Pub. No.: US 2022/0142497 A1
(43) Pub. Date: May 12, 2022

(54) ELECTROCARDIOGRAM LEAD ATTACHMENT DEVICE

(71) Applicant: Anthony Brunette, Oakdale, PA (US)

(72) Inventor: Anthony Brunette, Oakdale, PA (US)

(21) Appl. No.: 17/095,354

(22) Filed: Nov. 11, 2020

(51) Int. Cl. A61B 5/0428; A61B 5/0408

(52) U.S. Cl. CPC A61B 5/04286; A61B 2562/227; A61B 5/0408

(57) ABSTRACT

An electrocardiogram lead attachment device for an ECG, the electrocardiogram lead attachment device including a main body to be disposed on a body of a user, a plurality of lead attachment portions removably disposed on at least a portion of the main body to extend a distance away from the main body, and a plurality of lead connectors removably connected to at least a portion of at least one of the main body and the plurality of lead attachment portions to conduct an electrical current therethrough, such that an electrical potential difference may be measured between each of the plurality of lead connectors by the ECG.

### BACKGROUND

### 1. Field

The present general inventive concept relates generally to an attachment device, and particularly, to an electrocardiogram lead attachment device.

### 2. Description of the Related Art

An electrocardiogram (EKG or ECG) is a graph created by an ECG machine used to record electrical signals of the heart using electrodes in order to check for heart conditions during a heartbeat. An abnormality in an ECG pattern may indicate problems, such as atrial fibrillation and ventricular tachycardia. As such, the ECG provides medical professionals (i.e. doctors, nurses, medical technicians, etc.) with viable information as it relates to patient care and taking necessary steps to promote heart health.

Typically, the ECG uses ten electrodes to form twelve ECG leads, such that each ECG lead measures a specific electrical potential difference on different parts of a body of a patient. Unsurprisingly, the process of attaching ten electrodes to the patient can be time consuming and tedious, especially in emergency circumstances when the patient needs immediate care that requires ECG readings.

Therefore, there is a need for an electrocardiogram lead attachment device that simplifies attachment of ECG leads.

### SUMMARY

The present general inventive concept provides an electrocardiogram lead attachment device.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing an electrocardiogram lead attachment device for an ECG, the electrocardiogram lead attachment device including a main body to be disposed on a body of a user, a plurality of lead attachment portions removably disposed on at least a portion of the main body to extend a distance away from the main body, and a plurality of lead connectors removably connected to at least a portion of at least one of the main body and the plurality of lead attachment portions to conduct an electrical current therethrough, such that an electrical potential difference may be measured between each of the plurality of lead connectors by the ECG.

The main body may include a first side pair of elongated members, a second side pair of elongated members connected at a first end to a first end of the first side pair of elongated members, and a third side pair of elongated members connected at a first end to a second end of the second side pair of elongated members, and connected at a second end to a second end of the second side pair of elongated members.

The third side pair of elongated members may include a first section, and a second section separated from the first section, such that a space exists therebetween.

The main body may further include a connector portion to connect the first section to the second section.

The main body may be foldable.

The main body may fold in response to depressing a button.

The plurality of lead connectors may be positioned on the main body according to a conventional ECG layout.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates a rear perspective view of an electrocardiogram lead attachment device, according to an exemplary embodiment of the present general inventive concept; and

FIG. 2 illustrates a front perspective view of the electrocardiogram lead attachment device, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

Electrocardiogram Lead Attachment Device 100

Main Body 110

First Side Pair of Elongated Members 111

Second Side Pair of Elongated Members 112

Third Side Pair of Elongated Members 113

First Section 113a

Second Section 113b

Connector Portion 114

Fastener Receiving Apertures 120

Lead Attachment Portions 130

Fastener Receiving Apertures 131

Fasteners 140

Lead Connectors 150

Fastener Receiving Apertures 151

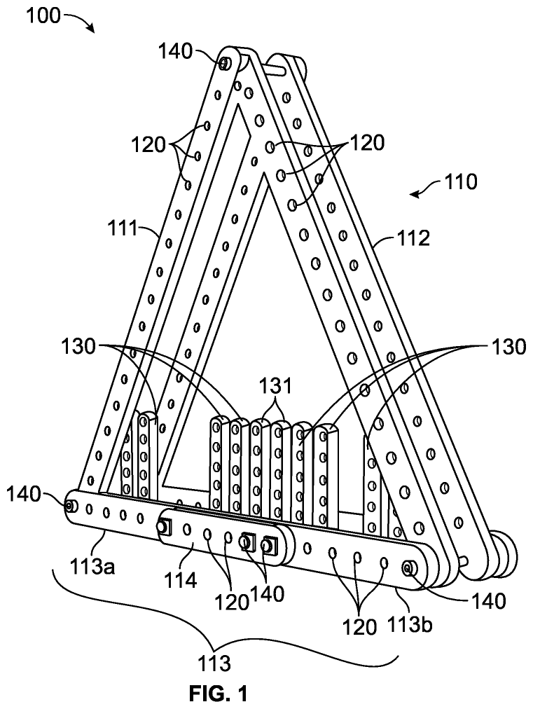

FIG. 1 illustrates a rear perspective view of an electrocardiogram lead attachment device 100, according to an exemplary embodiment of the present general inventive concept.

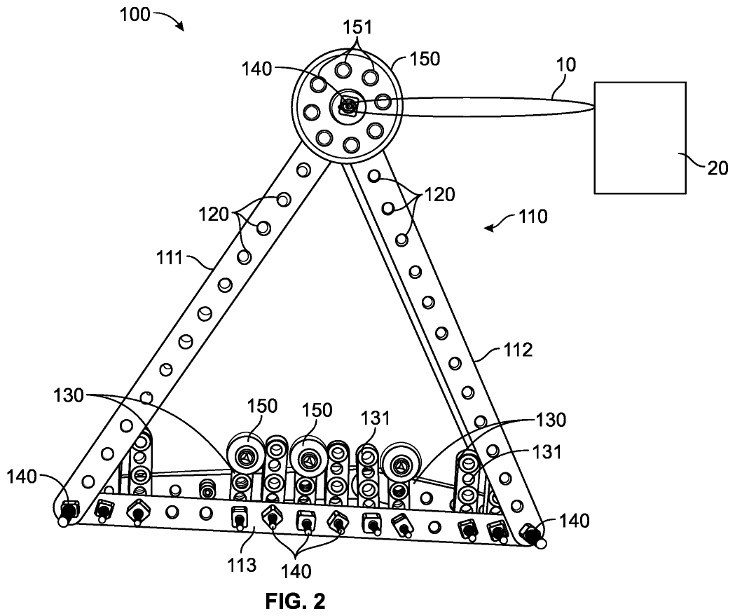

FIG. 2 illustrates a front perspective view of the electrocardiogram lead attachment device 100, according to an exemplary embodiment of the present general inventive concept.

The electrocardiogram lead attachment device 100 may be constructed from at least one of metal, plastic, and rubber, etc., but is not limited thereto.

The electrocardiogram lead attachment device 100 may include a main body 110, a plurality of fastener receiving apertures 120, a plurality of lead attachment portions 130, a plurality of fasteners 140, and a plurality of lead connectors 150, but is not limited thereto.

Referring to FIGS. 1 and 2, the main body 110 is illustrated to have a triangular shape. However, the main body 110 may be rectangular, circular, conical, pentagonal, hexagonal, heptagonal, octagonal, or any other shape known to one of ordinary skill in the art, but is not limited thereto.

The main body 110 may include a first side pair of elongated members 111, a second side pair of elongated members 112, a third side pair of elongated members 113, and a connector portion 114, but is not limited thereto.

Referring again to FIGS. 1 and 2, the first side pair of elongated members 111, the second side pair of elongated members 112, and the third side pair of elongated members 113 are illustrated to have an elliptical shape and/or may be flat rods. However, the first side pair of elongated members 111, the second side pair of elongated members 112, and/or the third side pair of elongated members 113 may be rectangular, circular, conical, pentagonal, hexagonal, heptagonal, octagonal, or any other shape known to one of ordinary skill in the art, but is not limited thereto.

The first side pair of elongated members 111, the second side pair of elongated members 112, and/or the third side pair of elongated members 113 may have equivalent dimensions (i.e. length, width, and/or height). As such, the main body 110 may be an equilateral triangle.

Moreover, each of the first side pair of elongated members 111, each of the second side pair of elongated members 112, and/or each of the third side pair of elongated members 113 may be a single elongated member and/or have more than two (i.e. more than a pair) elongated members depending on a preference of a user and/or manufacturer. In other words, the main body 110 may be customized.

A first end of the first side pair of elongated members 111 may be removably connected to a first end of the second side pair of elongated members 112. A second end of the second side pair of elongated members 111 may be removably connected to a first end of the third side pair of elongated members 113. A second end of the third side pair of elongated pair of members 113 may be removably connected to a second end of the first side pair of elongated members 111.

The third side pair of elongated members 113 may include a first section 113a and a second section 113b, but is not limited thereto.

Referring to FIG. 1, the third side pair of elongated members 113 is illustrated to be two separate pieces. However, each of the third side pair of elongated members 113 may be integral pieces similar to the first side pair of elongated members 111 and/or the second side pair of elongated members 112.

The first section 113a may be removably connected to the second end of the first side pair of elongated members 111. The second section 113b may be removably connected to the second end of the second side pair of elongated members 112. Additionally, a length of the first section 113a and/or a length of the second section 113b may be less than a length of the first side pair of elongated members 111 and/or a length of the second side pair of elongated members 112. In other words, the first section 113a may be separated from the second section 113b, such that a space exists therebetween.

The connector portion 114 may be connected at a first end to the first section 113a, and connected at a second end to the second section 113b. As such, the connector portion 114 may connect the first section 113a to the second section 113b. Additionally, the first end of the connector portion 114 and/or the second end of the connector portion 114 may move (i.e. rotate), toward and/or away from the first side pair of elongated members 111 and/or the second side pair of elongated members 112, such that the third side pair of elongated members 113 may bend, such that the main body 110 may be folded.

Also, each end of the first side pair of elongated members 111, each end of the second side pair of elongated members 112, and/or each end of the third side pair of elongated members 113 may move toward and/or away from each other, such that the main body 110 may be folded.

Optionally, a button may be disposed at each end of the first side pair of elongated members 111, the second side pair of elongated members 112, and/or the third side pair of elongated members 113 to allow the main body 110 to fold in response to being depressed.

The plurality of fastener receiving apertures 120 may be disposed on at least a portion of the main body 110. More specifically, the plurality of fastener receiving apertures 120 may be disposed on at least a portion of the first side pair of elongated members 111, the second side pair of elongated members 112, the third side pair of elongated members 113, and/or the connector portion 114.

Referring again to FIGS. 1 and 2, the plurality of lead attachment portions 130 may be removably connected between each of the first side pair of elongated members 111, each of the second side pair of elongated members 112, each of the third side pair of elongated members 113, and/or the connector portion 114.

Each of the plurality of lead attachment portions 130 may include a plurality of fastener receiving apertures 131, but is not limited thereto. Also, the plurality of lead attachment portions 130 may extend a predetermined distance away from the first side pair of elongated members 111, the second side pair of elongated members 112, the third side pair of elongated members 113, and/or the connector portion 114. For example, the plurality of lead attachment portions 130 may extend toward a center of the main body 110 between the first side pair of elongated members 111, the second side pair of elongated members 112, the third side pair of elongated members 113, and/or the connector portion 114.

Each of the plurality of fasteners 140 may include hooks and loops, a twine, a string, a rope, a magnet, a clasp, a hook, a screw, a nail, a bolt, a nut, a washer, and/or any combination thereof, but is not limited thereto.

Each of the plurality of fasteners 140 may be removably connected to at least one of the plurality of fastener receiving apertures 120 and/or the plurality of fastener receiving apertures 131. As such, the plurality of fasteners 140 may removably connect the first side pair of elongated members 111, the second side pair of elongated members 112, the third side pair of elongated members 113, and/or the connector portion 114 to each other. Furthermore, the plurality of fasteners 140 may removably connect the plurality of lead attachment portions 130 to the main body 110.

Each of the plurality of lead connectors 150 may include a plurality of fastener receiving apertures 151, but is not limited thereto.

The plurality of lead connectors 150 may be constructed to be an ECG lead, an ECG electrode, and/or any other type of electrically conductive pad. However, the plurality of lead connectors 150 may be constructed of any other type of material and/or shape based on the preference of the user and/or the manufacturer. Also, the plurality of lead connectors 150 may conduct and an electrical current therethrough, such that an electrical potential difference may be measured between each of the plurality of lead connectors 150 by an ECG 20.

Moreover, the plurality of lead connectors 150 may be removably connected to at least a portion of the main body 110 and/or the plurality of lead attachment portions 130 via the plurality of fasteners 140. In other words, the plurality of fasteners 140 may connect the plurality of lead receiving members 140 to at least a portion of the main body 110 and/or the plurality of lead attachment portions 130.

The plurality of lead connectors 150 may be positioned on the main body 110 to correspond to a predetermined layout and/or conventional ECG layout where conventional ECG leads are disposed on a body of the user (i.e. patient). In other words, the plurality of lead connectors 150 are disposed on the main body 110 and/or the plurality of lead attachment portions 130, such that the main body 110 may be disposed on the body of the user according to conventional ECG lead placement on the body of the user. For example, the conventional ECG leads may include bipolar limb leads (e.g., lead I RA, lead II LA, lead III LL), augmented unipolar limb leads (e.g., lead aVR, lead aVL, lead aVF), and unipolar chest leads (leads V1, V2, V3 and leads V4, V5, V6).

Furthermore, the plurality of fastener receiving apertures 151 may receive at least one of the plurality of fasteners 140 therein to connect another of the plurality of lead connectors 150 thereto. For example, at least one of the plurality of lead connectors 150 may be removably connected to another one of the plurality of lead connectors 150 to adjust a position thereof, such that the plurality of lead connectors 150 may correspond to conventional ECG lead placement on the body of the user.

Accordingly, the at least one of the plurality of lead connectors 150 may be connected to the ECG 20 using at least one ECG lead line 10.

Therefore, the electrocardiogram lead attachment device 100 may facilitate connection of an ECG machine to the body of the user by having a preconfigured arrangement of the plurality of lead connectors 150, such that placement of the

conventional ECG lead is not necessary. The present general inventive concept may include an electrocardiogram lead attachment device 100 for an ECG 20, the electrocardiogram lead attachment device 100 including a main body 110 to be disposed on a body of a user, a plurality of lead attachment portions 130 removably disposed on at least a portion of the main body 110 to extend a distance away from the main body 110, and a plurality of lead connectors 150 removably connected to at least a portion of at least one of the main body 110 and the plurality of lead attachment portions 130 to conduct an electrical current therethrough, such that an electrical potential difference may be measured between each of the plurality of lead connectors by the ECG 20.

The main body 110 may include a first side pair of elongated members 111, a second side pair 112 of elongated members connected at a first end to a first end of the first side pair of elongated members 111, and a third side pair of elongated members 113 connected at a first end to a second end of the second side pair of elongated members 112, and connected at a second end to a second end of the second side pair of elongated members 112.

The third side pair of elongated members 113 may include a first section 113a, and a second section 113b separated from the first section 113a, such that a space exists therebetween.

The main body 110 may further include a connector portion 114 to connect the first section 113a to the second section 113b.

The main body 110 may be foldable.

The main body 110 may fold in response to depressing a button.

The plurality of lead connectors 150 may be positioned on the main body 110 according to a conventional ECG layout.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. An electrocardiogram lead attachment device for an ECG, the electrocardiogram lead attachment device comprising:
a main body to be disposed on a body of a user;
a plurality of lead attachment portions removably disposed on at least a portion of the main body to extend a distance away from the main body; and
a plurality of lead connectors removably connected to at least a portion of at least one of the main body and the plurality of lead attachment portions to conduct an electrical current therethrough, such that an electrical potential difference may be measured between each of the plurality of lead connectors by the ECG.

2. The electrocardiogram lead attachment device of claim 1, wherein the main body comprises:
a first side pair of elongated members;
a second side pair of elongated members connected at a first end to a first end of the first side pair of elongated members; and
a third side pair of elongated members connected at a first end to a second end of the second side pair of elongated members, and connected at a second end to a second end of the second side pair of elongated members.

3. The electrocardiogram lead attachment device of claim 2, wherein the third side pair of elongated members comprises:
a first section; and
a second section separated from the first section, such that a space exists therebetween.

4. The electrocardiogram lead attachment device of claim 3, wherein the main body further comprises:
a connector portion to connect the first section to the second section.

5. The electrocardiogram lead attachment device of claim 1, wherein the main body is foldable.

6. The electrocardiogram lead attachment device of claim 5, wherein the main body folds in response to depressing a button.

7. The electrocardiogram lead attachment device of claim 1, wherein the plurality of lead connectors are positioned on the main body according to a conventional ECG layout.
